(19) United States
(12) Patent Application Publication
(10) Pub. No.: US 2022/0024976 A1
(43) Pub. Date: Jan. 27, 2022

(54) SALT AND CRYSTALLINE FORMS OF RAPASTINEL

(71) Applicant: NAUREX INC., Madison, NJ (US)

(72) Inventor: Ke WU, Irvine, CA (US)

(21) Appl. No.: 17/421,915

(22) PCT Filed: Jan. 13, 2020
(86) PCT No.: PCT/US2020/013327

§ 371 Date: Jul. 9, 2021

Related U.S. Application Data
(60) Provisional application No. 62/791,377, filed on Jan. 11, 2019.

(51) Int. Cl. C07K 5/103; A61K 45/06; A61K 38/07

(52) U.S. Cl. CPC C07K 5/1013; A61K 38/07; A61K 45/06

(57) ABSTRACT

The application relates to salts of a compound of rapastinel, pharmaceutical compositions thereof, and uses of the salt forms for the treatment of cognitive and neurological diseases and disorders: (Formula I).

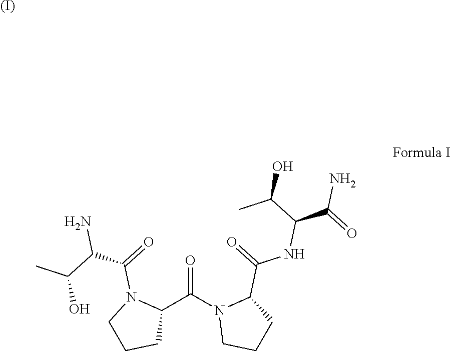

### FIELD

The application is related to rapastinel, its salt forms and their use in the treatment of neurological and cognitive diseases and disorders.

### BACKGROUND

An N-methyl-D-aspartate (NMDA) receptor is a postsynaptic, ionotropic receptor that is responsive to, inter alia, the excitatory amino acids glutamate and glycine and the synthetic compound NMDA. The NMDA receptor (NMDAR) appears to control the flow of both divalent and monovalent ions into the postsynaptic neural cell through a receptor associated channel and has drawn particular interest since it appears to be involved in a broad spectrum of CNS disorders. The NMDAR has been implicated, for example, in neurodegenerative disorders including stroke-related brain cell death, convulsive disorders, and learning and memory. NMDAR also plays a central role in modulating normal synaptic transmission, synaptic plasticity, and excitotoxicity in the central nervous system. The NMDAR is further involved in Long-Term Potentiation (LTP), which is the persistent strengthening of neuronal connections that underlie learning and memory The NMDAR has been associated with other disorders ranging from hypoglycemia and cardiac arrest to epilepsy. In addition, there are preliminary reports indicating involvement of NMDA receptors in the chronic neurodegeneration of Huntington's, Parkinson's, and Alzheimer's diseases. Activation of the NMDA receptor has been shown to be responsible for post-stroke convulsions, and, in certain models of epilepsy, activation of the NMDA receptor has been shown to be necessary for the generation of seizures. In addition, certain properties of NMDA receptors suggest that they may be involved in the information-processing in the brain that underlies consciousness itself. Further, NMDA receptors have also been implicated in certain types of spatial learning.

In view of the association of NMDAR with various disorders and diseases, NMDA-modulating small molecule agonist and antagonist compounds have been developed for therapeutic use. NMDA receptor compounds may exert dual (agonist/antagonist) effect on the NMDA receptor through the allosteric sites. These compounds are typically termed “partial agonists”. In the presence of the principal site ligand, a partial agonist will displace some of the ligand and thus decrease Ca⁺⁺ flow through the receptor. In the absence of the principal site ligand or in the presence of a lowered level of the principal site ligand, the partial agonist acts to increase Ca⁺⁺ flow through the receptor channel.

Recently, a partial agonist of NMDAR with the following structure has been reported (rapastinel or GLYX-13):

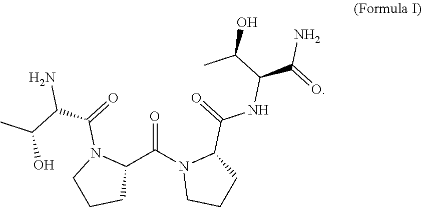

PCT/US2017/015851 describes a process for synthesis of peptide compounds, including rapastinel. New salt and crystalline forms as well as methods for preparing them can have practical and industrial advantages.

### SUMMARY

This disclosure relates to salts of a compound of Formula I (rapastinel or GLYX-13) as described herein, pharmaceutical compositions thereof as described herein, and uses thereof as described herein.

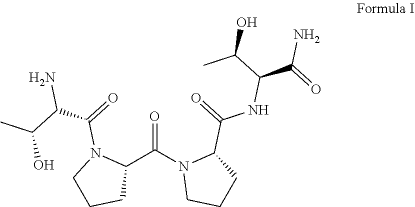

In one aspect, provided herein are tartrate, succinate and hydrochloric salts of rapastinel. Methods of making and using these salts are also provided. Also provided are salts of rapastinel obtained by the processes described herein (e.g., obtained by the described methods of making). Pharmaceutical compositions comprising one or more salts of rapastinel, and a pharmaceutically acceptable carrier are provided. Articles of manufacture and unit dosage forms comprising one or more salts of rapastinel are provided. Kits comprising one or more salts of rapastinel and instructions for use (e.g., instructions for use in a central nervous system disorder) are also provided.

### FIGURES

FIG. 1: X-ray diffraction pattern of a polymorph form of the tartrate salt rapastinel.

FIG. 2: Thermogravimetric analysis of a tartrate salt polymorph of rapastinel.

FIG. 3: Differential scanning calorimetry (DSC) curve of a tartrate salt polymorph of rapastinel.

FIG. 4: X-ray diffraction pattern of a polymorph form of the succinate salt rapastinel.

FIG. 5: Thermogravimetric analysis of a succinate salt polymorph of rapastinel.

FIG. 6: Differential scanning calorimetry (DSC) curve of a succinate salt polymorph of rapastinel.

FIG. 7: X-ray diffraction pattern of a polymorph form of the hydrochloride salt rapastinel.

FIG. 8: Thermogravimetric analysis of a hydrochloride salt polymorph of rapastinel.

FIG. 9: Differential scanning calorimetry (DSC) curve of a hydrochloride salt polymorph of rapastinel.

### DESCRIPTION

In some embodiments, a tartrate salt of a compound of Formula I (rapastinel) is provided by this disclosure. In some embodiments, a hydrochloride salt of rapastinel is provided by this disclosure. In some embodiments, a succinate salt of rapastinel is provided by this disclosure.

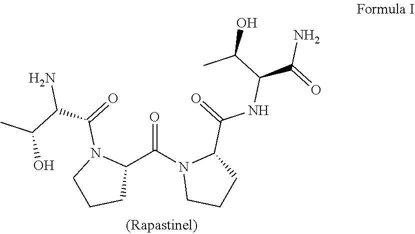

In one aspect, provided herein are tartrate salt of a compound of Formula I:

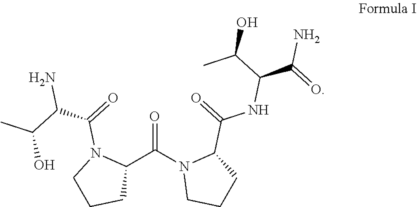

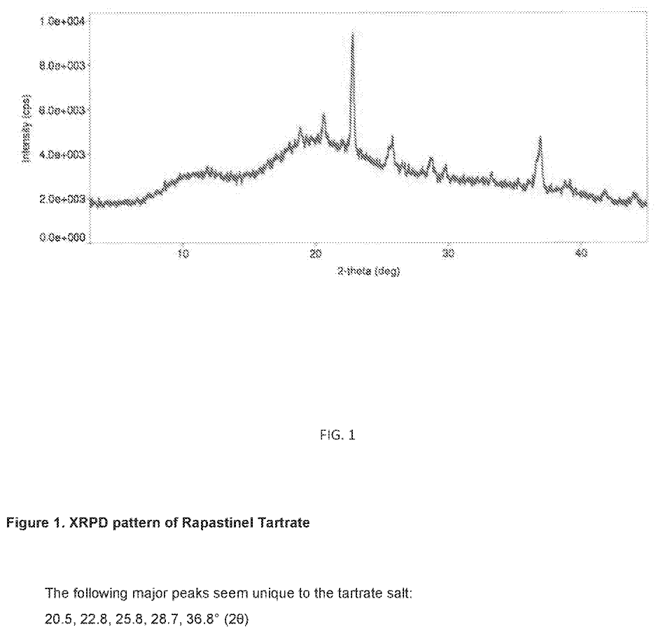

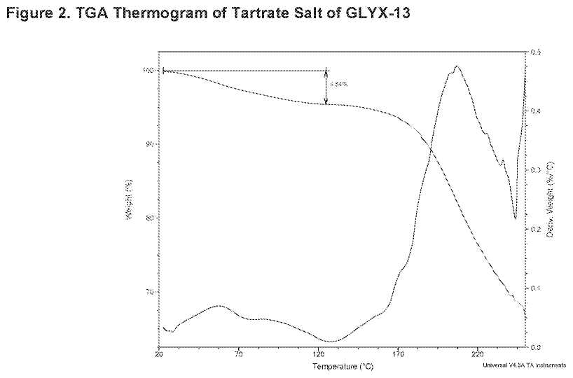

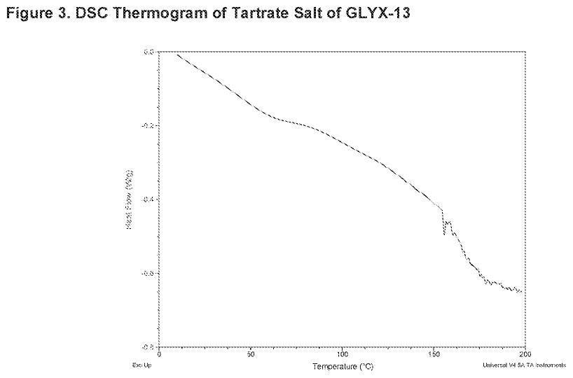

Additionally, provided are pharmaceutical compositions comprising the tartrate salt of a compound of Formula I and a pharmaceutical carrier, excipient, adjuvant, or vehicle. Polymorphic forms of the tartrate salt of compound of Formula I are also provided. In one aspect, the polymorphic form is characterized by an X-ray diffraction pattern comprising (2θ) reflections, plus or minus 0.2 degrees (2θ) at 20.5, 22.8, 25.8, 28.7, 36.8. Provided herein are polymorphic forms of the tartrate salts of a compound of Formula I. The polymorphic form can be characterized by an X-ray diffraction pattern substantially as shown in FIG. 1; or characterized by thermogravimetric analysis (TGA) comprising a thermogram substantially the same as shown in FIG. 2; or characterized by a differential scanning calorimetry (DSC) curve substantially the same as shown in FIG. 3. In one aspect, provided are pharmaceutical compositions comprising the polymorphic forms and a pharmaceutical carrier, excipient, adjuvant, or vehicle.

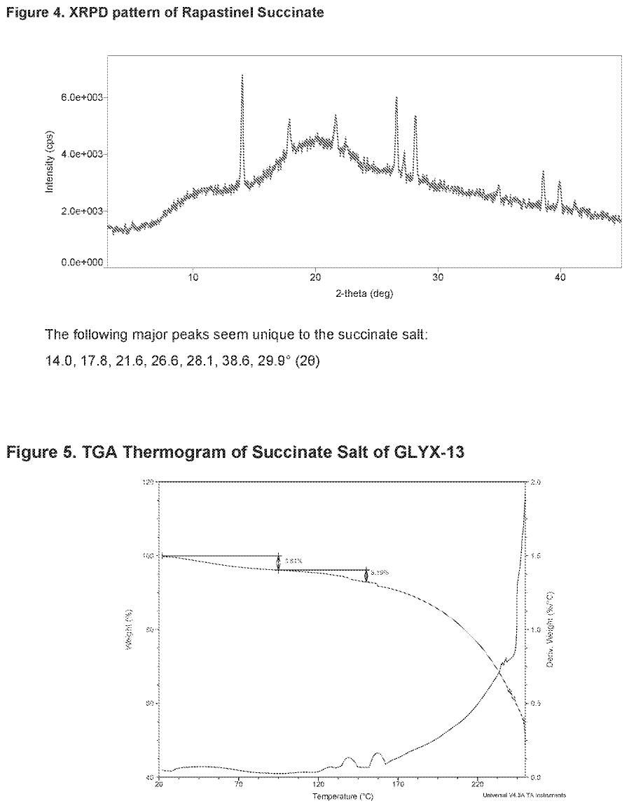

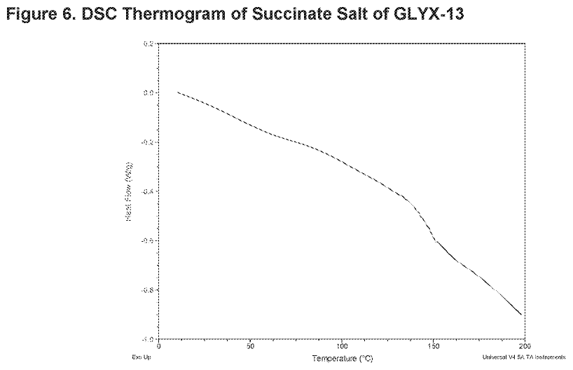

Additionally, provided are pharmaceutical compositions comprising the succinate salt of a compound of Formula I and a pharmaceutical carrier, excipient, adjuvant, or vehicle. Polymorphic forms of the succinate salt of compound of Formula I are also provided. In one aspect, the polymorphic form is characterized by an X-ray diffraction pattern comprising (2θ) reflections, plus or minus 0.2 degrees (2θ) at 14.0, 17.8, 21.6, 26.6, 28.1, 38.6, 29.9. Provided herein are polymorphic forms of the succinate salts of a compound of Formula I. The polymorphic form can be characterized by an X-ray diffraction pattern substantially as shown in FIG. 4; or characterized by thermogravimetric analysis (TGA) comprising a thermogram substantially the same as shown in FIG. 5; or characterized by a differential scanning calorimetry (DSC) curve substantially the same as shown in FIG. 6. In one aspect, provided are pharmaceutical compositions comprising the polymorphic forms and a pharmaceutical carrier, excipient, adjuvant, or vehicle.

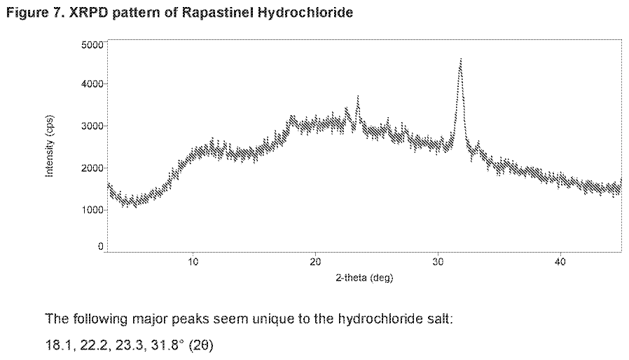

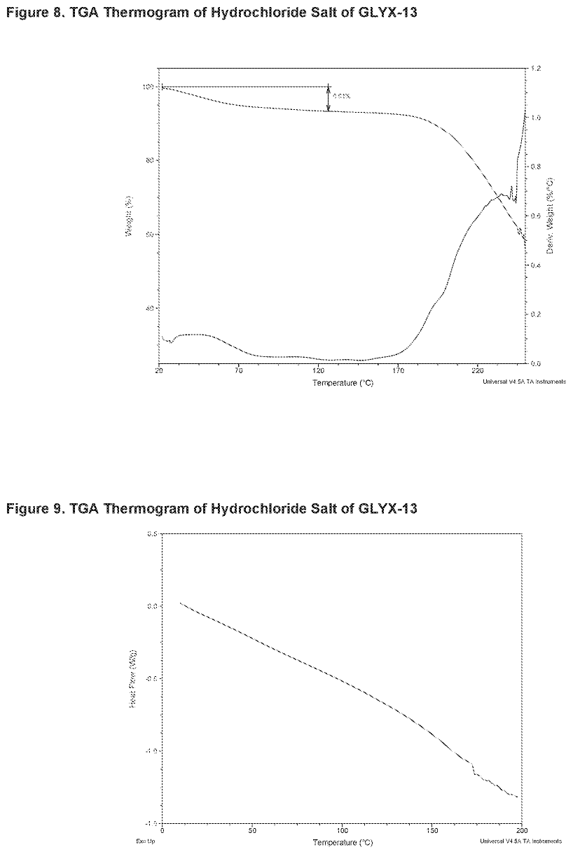

Additionally, provided are pharmaceutical compositions comprising the hydrochloric salt of a compound of Formula I and a pharmaceutical carrier, excipient, adjuvant, or vehicle. Polymorphic forms of the hydrochloric salt of compound of Formula I are also provided. In one aspect, the polymorphic form is characterized by an X-ray diffraction pattern comprising (2θ) reflections, plus or minus 0.2 degrees (2θ) at 18.1, 22.2, 23.3, 31.8. Provided herein are polymorphic forms of the hydrochloric salts of a compound of Formula I. The polymorphic form can be characterized by an X-ray diffraction pattern substantially as shown in FIG. 7; or characterized by thermogravimetric analysis (TGA) comprising a thermogram substantially the same as shown in FIG. 8; or characterized by a differential scanning calorimetry (DSC) curve substantially the same as shown in FIG. 9. In one aspect, provided are pharmaceutical compositions comprising the polymorphic forms and a pharmaceutical carrier, excipient, adjuvant, or vehicle.

In one aspect provided are methods for treating cognitive, neurological or psychological disease or disorder by administration of a therapeutically effective amount of tartrate, succinate or hydrochloride salt of a compound of Formula I, preferably a particular polymorphic form described herein.

In some embodiments, the cognitive, neurological or psychological disease or disorder is selected from the group consisting of deficiency in memory, intellect, or learning and logic ability; reduction in any particular individual's functioning in one or more cognitive aspects; age-related cognitive decline; dementia; Alzheimer's disease; multi-infarct dementia; alcoholic dementia or other drug-related dementia; dementia associated with intracranial tumors or cerebral trauma; dementia associated with Huntington's disease or Parkinson's disease; AIDS-related dementia; delirium; amnestic disorder; mental retardation; a learning disorder including reading disorder, mathematics disorder, or a disorder of written expression; attention-deficit/hyperactivity disorder; schizophrenia, schizophrenia including negative symptoms; schizophreniform disorder; schizoaffective disorder, schizoaffective disorder of the delusional type, schizoaffective disorder of the depressive type; delusional disorder; substance-induced psychotic disorder; personality disorder of the paranoid type; personality disorder of the schizoid type; panic disorder; phobias: obsessive-compulsive disorder; stress disorders; generalized anxiety disorder; movement disorders involving Huntington's disease; dyskinesia associated with dopamine agonist therapy: Parkinson's disease: restless leg syndrome; disorders comprising as a symptom thereof a deficiency in cognition.

In some embodiments, the cognitive, neurological or psychological disease or disorder is selected from depression, major depressive disorder, refractory depression, pre-menstrual dysphoric disorder, post-partum depression, acute depressive episodes with bipolar I, treatment resistant depression, general anxiety disorder, obsessive compulsive disorder, panic disorder, post-traumatic stress disorder, social anxiety disorder, bulimia nervosa, cognitive dysfunction in pre-menstrual dysphoric disorder, attention deficit hyperactivity disorder, attention deficit hyperactivity disorder in adult patients, and combinations thereof.

In some embodiments, the cognitive, neurological or psychological disease or disorder is suicidality, or suicidal ideation.

In some embodiments, the cognitive, neurological or psychological disease or disorder is depression, major depressive disorder, post-partum depression or post-traumatic stress disorder.

In some embodiment, the patient is undergoing treatment with one or more additional agents selected from selective serotonin reuptake inhibitors (SSRI), serotonin agonists, antagonists and modulators, selective norepinephrine reuptake inhibitors (SNRIs). In some embodiments, the patient is undergoing treatment with one or more additional agents selected from opiate agonists, opiate antagonists, opiate partial agonists, calcium channel antagonists, 5HT, 5-HT_{1A} complete or partial receptor agonists or antagonists, 5-HT_{2A} complete or partial receptor agonists or antagonists, 5-HT₃ complete or partial receptor agonists or antagonists, sodium channel antagonists, N-methyl-D-aspartate (NMDA) receptor antagonists, COX-2 selective inhibitors, neurokinin receptor 1 (NK1) antagonists, non-steroidal anti-inflammatory drugs (NSAID), selective serotonin reuptake inhibitors (SSRI) and/or selective serotonin and norepinephrine reuptake inhibitors (SSNRI), tricyclic antidepressant drugs, norepinephrine modulators, lithium, valproate, norepinephrine reuptake inhibitors, monoamine oxidase inhibitors (MAOIs), reversible inhibitors of monoamine oxidase (RIMAs), alpha-adrenoreceptor antagonists, atypical anti-depressants, benzodiazepines, corticotropin releasing factor (CRF) antagonists, Neurontin (gabapentin) and pregabalin.

In some embodiments, the patient is undergoing treatment with one more additional agents selected from a selective serotonin reuptake inhibitor (SSRI), serotonin-norepinephrine reuptake inhibitor (SNRI), serotonin modulator and stimulator (SMS), serotonin antagonist and reuptake inhibitor (SARI), norepinephrine reuptake inhibitor (NRI), norepinephrine-dopamine reuptake inhibitor (NDRI), tricyclic antidepressant (TCA), tetracyclic antidepressant (TeCA), monoamine oxidase inhibitor (MAOI) and atypical antipsychotic.

### Definitions

As used herein, the words or terms set forth below have the following definitions:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a pharmaceutical composition to a subject, or alternatively a subject receiving a pharmaceutical composition.

“Treating” includes any effect, e.g., lessening, reducing, modulating, or eliminating, that results in the improvement of the condition, disease, disorder and the like. “Individual,” “patient,” or “subject” are used interchangeably and include any animal, including mammals, preferably mice, rats, other rodents, rabbits, dogs, cats, swine, cattle, sheep, horses, or primates, and most preferably humans.

The term “effective amount” refers to an amount of the subject component, e.g., GLYX-13 (or a composition containing GLYX-13) that will elicit the biological or medical response of a tissue, system, animal or human that is being sought by the researcher, veterinarian, medical doctor or other clinician.

“Patient” means a human or non-human subject receiving medical or veterinary care. Accordingly, the compositions as disclosed herein can be used in treating any animal, such as, for example, mammals, or the like.

“Pharmaceutical composition” means a composition comprising an active pharmaceutical ingredient, such as, for example, a CGRP antagonist, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

The constituent ingredients of a pharmaceutical composition can be included in a single composition (that is, all the constituent ingredients, except for any required reconstitution fluid, are present at the time of initial compounding of the pharmaceutical composition) or as a two-component system, for example a vacuum-dried composition reconstituted with a reconstitution vehicle which can, for example, contain an ingredient not present in the initial compounding of the pharmaceutical composition. A two-component system can provide several benefits, including that of allowing incorporation of ingredients which are not sufficiently compatible for long-term shelf storage with the first component of the two-component system. A pharmaceutical composition can also include preservative agents such as benzyl alcohol, benzoic acid, phenol, parabens and sorbic acid. Pharmaceutical compositions can include, for example, excipients, such as surface active agents; dispersing agents; inert diluents; granulating and disintegrating agents; binding agents; lubricating agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; antioxidants; stabilizing agents; and pharmaceutically acceptable polymeric or hydrophobic materials and other ingredients known in the art and described, for example in Genaro, ed., 1985, Remington's Pharmaceutical Sciences, Mack Publishing Co., Easton, Pa., which is incorporated herein by reference.

In some embodiments, a therapeutically effective amount of GLYX-13 for adult human treatment administered, for example, during an induction period of time, are in the range of about 0.01 mg/kg to about 1000 mg/kg per administration (e.g., about 0.01 mg/kg to about 100 mg/kg, about 0.01 mg/kg to about 50 mg/kg, about 0.01 mg/kg to about 25 mg/kg, about 0.01 mg/kg to about 10 mg/kg, about 0.1 mg/kg to about 100 mg/kg, about 0.1 mg/kg to about 50 mg/kg, about 0.1 mg/kg to about 50 mg/kg, about 0.1 mg/kg to about 10 mg/kg, about 1 mg/kg to about 100 mg/kg, about 1 mg/kg to about 50 mg/kg, about 1 mg/kg to about 50 mg/kg per day, about 1 mg/kg to about 10 mg/kg, or about 1 mg/kg to about 10 mg/kg per administration, e.g., once a week, twice a week or three times a week and/or as described herein). The dosage of GLYX-13 may be at any dosage including, but not limited to, about 1 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 2.5 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg. In certain embodiments, GLYX-13 may be therapeutically effective for depression with a range (e.g., an intravenous dose range) of about 1 to about 10 mg/kg, e.g., about 5 to about 10 mg/kg, e.g. about 1 mg/kg, about 5 mg/kg, or about 10 mg/kg.

In some embodiments, a therapeutically effective amount of GLYX-13 for adult human treatment administered, for example, during an induction period (administration period) of time may be a fixed dose of about 1000 mg to about 200 mg, or 900 mg to about 100 mg e.g., about 200 mg to about 500 mg, e.g., 50 mg, 100 mg, 225 mg, 250 mg, 200 mg, 300 mg, 350 mg, 450 mg, 500 mg, 600 mg, 700 mg, 750 mg, and/or 900 mg unit dose. It will be appreciated that a maintenance dose may be lower than the induction dose.

In some embodiments, any of the GLYX-13 dosages described herein can be administered on a less than daily basis, e.g., every other day (e.g., every two days); one or two times a week; one, two or three times a week; two or three times a week; twice weekly (e.g. every 3 days, every 4 days, every 5 days, every 6 days or e.g. administered with an interval of about 2 to about 3 days between doses); every three to four days; once a week; once every two weeks (bi-weekly); twice monthly; once a month or even less often. In certain embodiments, GLYX-13 is administered at a frequency of once a week, twice a week, once every two weeks, or any combination thereof.

In certain embodiments GLYX-13 (rapastinel) is administered at a range (e.g., an intravenous dose range) of about 1 to about 10 mg/kg, e.g., about 5 to about 10 mg/kg, e.g. about 1 mg/kg, about 5 mg/kg, or about 10 mg/kg, and/or GLYX-13 is administered at a frequency of once a week, once every two weeks, or any combination thereof.

In some embodiments, the methods and regimens include two or more treatment cycles (e.g. continuous cycles), in which each cycle includes an induction period of time and a rest period of time. As the skilled person will appreciate, each of the treatment cycles can be independently varied from one another in terms of dosage, frequency, duration of induction period of time, duration of rest period of time, etc.

### Administration and Formulations

GLYX-13 as well as any other pharmacological agent (e.g., one or more other antidepressant agents) of the present invention may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations, or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In some embodiments, GLYX-13 herein may be administered parenterally to a patient including, but not limited to, subcutaneously, intramuscularly, and intravenously. In some embodiments, one or more of the components of the combinations described herein may also be administered via slow controlled i.v. infusion or by release from an implant device.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, intranasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, general safety and purity standards as required by FDA Office of Biologics standards. The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The combinations described herein may also contain other active compounds providing supplemental, additional, or enhanced therapeutic functions. Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

Disclosed compounds may be provided as part of a liquid or solid formulation, for example, aqueous or oily suspensions, solutions, emulsions, syrups, and/or elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl hydroxybenzoate and sorbic acid. Contemplated compounds may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water. For example, pharmaceutical formulations of rapastinel disclosed in U.S. Patent Publication Nos. 20170296616 and 20170049844, incorporated by reference herein, can be used with the salt and polymorphic forms of the instant application.

The present invention has multiple aspects, illustrated by the following non-limiting examples.

### EXAMPLES

The following non-limiting examples provide those of ordinary skill in the art with possible case scenarios and specific methods to treat conditions within the scope of the present disclosure and are not intended to limit the scope of the disclosure.

### Example 1

### 1) Hydrochloride Salt

Drug substance (0.5 g GLYX-13 base) was dissolved in 0.5 mL methanol in a 20 mL scintillation glass vial by using heating with heating gun. A light hazy solution was formed. To the vial 0.5 mL 3N hydrochloride acid (HCl) in methanol was added. The mixture was vortexed at 3000 rpm for about 5 minutes. At ambient temperature, resulting solution was added drop-wise to 10 mL acetone and was magnetically stirred at 1000 rpm. A white suspension formed. Another 4 mL acetone was added to the suspension and continued stirring for 3 days at room temperature (RT). The suspension was vacuum-filtered through a polytetrafluoroethylene (PTFE) membrane under nitrogen (N₂) purging. The solids were rinsed on the filter membrane with 20 mL of acetone. The isolated solids were placed under vacuum at room temperature (22±2° C.) for 1 day.

### Example 2: Acetate Salt

GLYX-13 base (0.3 g) was dissolved in 0.7 mL acetic acid in a 20 mL scintillation glass vial and stirred at room temperature for 30 minutes. The resulting solution was drop-wise to 11 mL 2-methyltetrahydrofuran with magnetic stirring at 1000 rpm. The stirring was continued for 3 days at RT. The suspension was vacuum-filtered through a PTFE membrane under N₂ purging. The solids were rinsed on the filter membrane with 15 mL of 2-methyltetrahydrofuran. The isolated solids were placed under vacuum at RT for 1 day.

### Example 3: Succinate Salt

GLYX-13 base (0.53 g) was dissolved in 0.53 mL methanol in a 20 mL scintillation glass vial by using heating with heating gun. A light hazy solution was formed. In a separate vial 0.19 g succinic acid dissolved in 0.5 mL methanol. The two solutions were mixed for 45 minutes at ambient temperature. The resulting solution was added drop-wise to 20 mL methyl tertiary-butyl ether (MTBE) magnetically stirred at 1000 rpm. Stirring was continued for overnight at RT. A white concentrated suspension formed. The suspension was vacuum filtered through a PTFE membrane under N₂ purging. The solids were rinsed on the filter membrane with 20 mL of MTBE. The isolated solids under placed under vacuum at RT for 1 day.

### Example 4 L-Tartrate Salt

GLYX-13 base (0.7 g) was dissolved in 0.7 mL methanol in a 20 mL scintillation glass vial by using heating with heating gun. A light hazy solution was formed. In a separate vial, 0.31 g L-tartaric acid was dissolved in 0.7 mL methanol. The above two solutions were mixed for 45 minutes at ambient temperature. The resulting solution was added drop-wise to 23 mL acetone magnetically stirred at 1000 rpm with continued stirring for overnight at RT. A white concentrated suspension was formed. The suspension was vacuum-filtered through a PTFE membrane under N₂ purging. The solids were rinsed on the filter membrane with 20 mL of acetone. The isolated solids were placed under vacuum at RT for 1 day.

The above salt forms of GLYX-13 can be converted to free base form by the following steps:

(1) Dissolve the peptide salt in an appropriate solvent system (e.g., methanol, water, acetonitrile, or their mixture, etc., depending on the solubility of the peptide salt in respective systems).

(2) Add suitable base (such as sodium carbonate, sodium bicarbonate, bicarbonate resin, etc.) at appropriate stoichiometric ratio relative to the salt.

(3) Mix the system by suitable agitation method at appropriate temperature for adequate period of time.

(4) Isolate the resulting freebase by suitable operations such as filtration or extraction followed by evaporation or in vacuo drying.

1. Tartrate salt of a compound of Formula I:

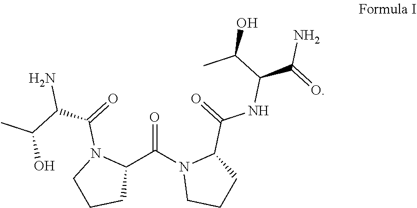

2. A pharmaceutical composition comprising said tartrate salt of a compound of Formula I according to claim 1 and a pharmaceutical carrier, excipient, adjuvant, or vehicle.

3. Polymorph form of a tartrate salt of a compound of Formula I 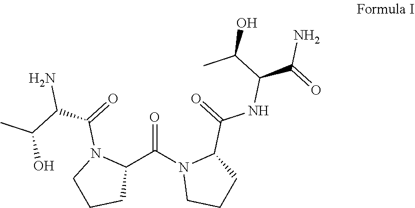
Wherein the polymorphic form is characterized by; an X-ray diffraction pattern comprising (2θ) reflections, plus or minus 0.2 degrees (2θ) at 20.5, 22.8, 25.8, 28.7, 36.8; or an X-ray diffraction pattern substantially as shown in FIG. 1.

4. Polymorph form of a tartrate salt of a compound of Formula I 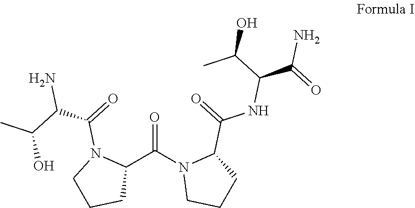
Characterized by thermogravimetric analysis (TGA) comprising a thermogram substantially the same as shown in FIG. 2.

5. Polymorph form of a tartrate salt of a compound of Formula I 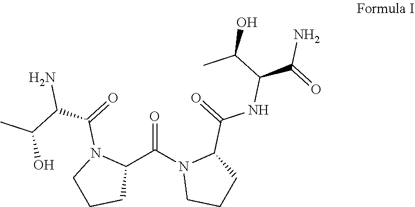
Characterized by a differential scanning calorimetry (DSC) curve substantially the same as shown in FIG. 3.

6. A pharmaceutical composition comprising said polymorphic form of claim 3, 4 or 5 and a pharmaceutical carrier, excipient, adjuvant, or vehicle.

7. Succinate salt of a compound of Formula I:

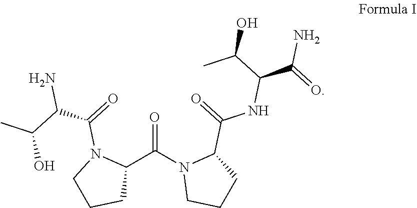

8. A pharmaceutical composition comprising said succinate salt of a compound of Formula I according to claim 7 and a pharmaceutical carrier, excipient, adjuvant, or vehicle.

9. Polymorph form of a succinate salt of a compound of Formula I 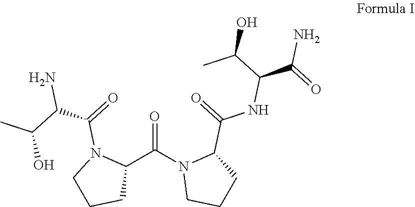
Wherein the polymorphic form is characterized by; an X-ray diffraction pattern comprising (2θ) reflections, plus or minus 0.2 degrees (2θ) at 14.0, 17.8, 21.6, 26.6, 28.1, 38.6, 29.9; or an X-ray diffraction pattern substantially as shown in FIG. 4.

10. Polymorph form of a succinate salt of a compound of Formula I 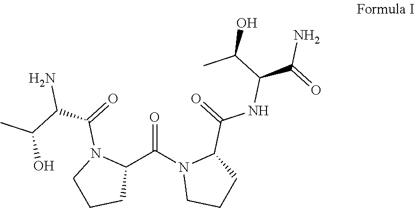
Characterized by thermogravimetric analysis (TGA) comprising a thermogram substantially the same as shown in FIG. 5.

11. Polymorph form of a succinate salt of a compound of Formula I 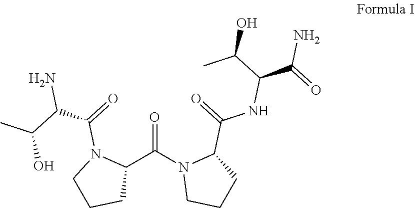
Characterized by a differential scanning calorimetry (DSC) curve substantially the same as shown in FIG. 6.

12. A pharmaceutical composition comprising said polymorphic form of claim 9, 10 or 11 and a pharmaceutical carrier, excipient, adjuvant, or vehicle.

13. Hydrochloride salt of a compound of Formula I:

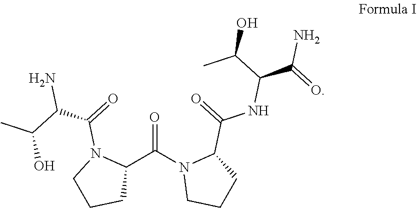

14. A pharmaceutical composition comprising said hydrochloride salt of a compound of Formula I according to claim 13 and a pharmaceutical carrier, excipient, adjuvant, or vehicle.

15. Polymorph form of a hydrochloride salt of a compound of Formula I 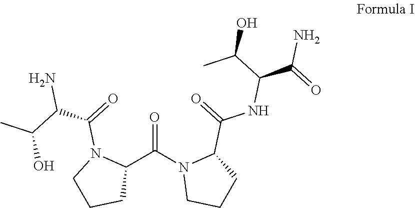
Wherein the polymorphic form is characterized by; an X-ray diffraction pattern comprising (2θ) reflections, plus or minus 0.2 degrees (2θ) at 18.1, 22.2, 23.3, 31.8; or an X-ray diffraction pattern substantially as shown in FIG. 7.

16. Polymorph form of a hydrochloride salt of a compound of Formula I 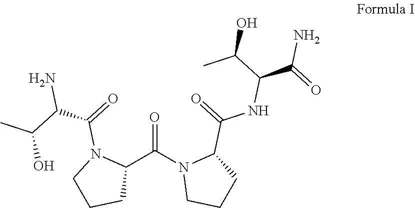
Characterized by thermogravimetric analysis (TGA) comprising a thermogram substantially the same as shown in FIG. 8.

17. Polymorph form of a hydrochloride salt of a compound of Formula I 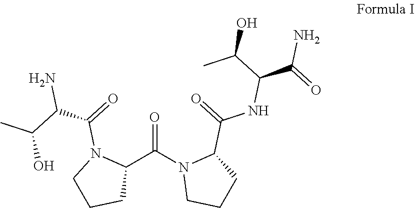
Characterized by a differential scanning calorimetry (DSC) curve substantially the same as shown in FIG. 9.

18. A pharmaceutical composition comprising said polymorphic form of claim 15, 16 or 17 and a pharmaceutical carrier, excipient, adjuvant, or vehicle.

19. A method of treating a patient suffering from a cognitive, neurological or psychological disease or disorder comprising the step of administering to said patient a therapeutically effective amount of tartrate, succinate or hydrochloride salt of a compound of Formula I

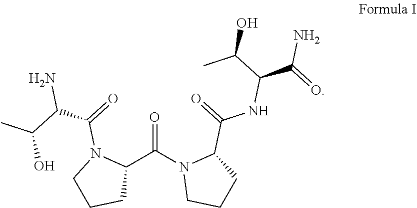

20. A method of treating a patient suffering from a cognitive, neurological or psychological disease or disorder comprising the step of administering to said patient a therapeutically effective amount of a tartrate, succinate or hydrochloride salt of a compound of Formula I, according to any of claims 1-18.

21. The method according to any of claims 19-20 wherein said cognitive, neurological or psychological disease or disorder is selected from the group consisting of deficiency in memory, intellect, or learning and logic ability; reduction in any particular individual's functioning in one or more cognitive aspects; age-related cognitive decline; dementia; Alzheimer's disease; multi-infarct dementia; alcoholic dementia or other drug-related dementia; dementia associated with intracranial tumors or cerebral trauma; dementia associated with Huntington's disease or Parkinson's disease; AIDS-related dementia; delirium; amnestic disorder; mental retardation; a learning disorder including reading disorder, mathematics disorder, or a disorder of written expression; attention-deficit/hyperactivity disorder; schizophrenia, schizophrenia including negative symptoms; schizophreniform disorder; schizoaffective disorder, schizoaffective disorder of the delusional type, schizoaffective disorder of the depressive type; delusional disorder; substance-induced psychotic disorder; personality disorder of the paranoid type; personality disorder of the schizoid type; panic disorder; phobias; obsessive-compulsive disorder; stress disorders; generalized anxiety disorder; movement disorders involving Huntington's disease; dyskinesia associated with dopamine agonist therapy: Parkinson's disease: restless leg syndrome; disorders comprising as a symptom thereof a deficiency in cognition.

22. The method according to any of claims 19-20 wherein said disease or disorder is selected from depression, major depressive disorder, refractory depression, pre-menstrual dysphoric disorder, post-partum depression, acute depressive episodes with bipolar I, treatment resistant depression, general anxiety disorder, obsessive compulsive disorder, panic disorder, post-traumatic stress disorder, social anxiety disorder, bulimia nervosa, cognitive dysfunction in pre-menstrual dysphoric disorder, attention deficit hyperactivity disorder, attention deficit hyperactivity disorder in adult patients, and combinations thereof.

23. The method according any of claims 19-21, wherein said disease or disorder is suicidality, or suicidal ideation.

24. The method according to any of claims 19-21, wherein said disease or disorder is depression.

25. The method according to any of claims 19-21, wherein said disease or disorder is major depressive disorder.

26. The method according to any of claims 19-21, wherein said disease or disorder is post-partum depression.

27. The method according to any of claims 19-21, wherein said disease or disorder is post-traumatic stress disorder.

28. The method according to any of claims 19-21, wherein said patient is undergoing treatment with one or more additional agents selected from selective serotonin reuptake inhibitors (SSRI), serotonin agonists, antagonists and modulators, selective norepinephrine reuptake inhibitors (SNRIs).

29. The method according to any of claims 19-21 wherein said patient is undergoing treatment with one or more additional agents selected from opiate agonists, opiate antagonists, opiate partial agonists, calcium channel antagonists, 5HT, 5-HT_{1A} complete or partial receptor agonists or antagonists, 5-HT_{2A} complete or partial receptor agonists or antagonists, 5-HT₃ complete or partial receptor agonists or antagonists, sodium channel antagonists, N-methyl-D-aspartate (NMDA) receptor antagonists, COX-2 selective inhibitors, neurokinin receptor 1 (NK1) antagonists, non-steroidal anti-inflammatory drugs (NSAID), selective serotonin reuptake inhibitors (SSRI) and/or selective serotonin and norepinephrine reuptake inhibitors (SSNRI), tricyclic antidepressant drugs, norepinephrine modulators, lithium, valproate, norepinephrine reuptake inhibitors, monoamine oxidase inhibitors (MAOIs), reversible inhibitors of monoamine oxidase (RIMAs), alpha-adrenoreceptor antagonists, atypical anti-depressants, benzodiazepines, corticotropin releasing factor (CRF) antagonists, Neurontin (gabapentin) and pregabalin.

30. The method according to any of claims 19-21 wherein said patient is undergoing treatment with one more additional agents selected from a selective serotonin reuptake inhibitor (SSRI), serotonin-norepinephrine reuptake inhibitor (SNRI), serotonin modulator and stimulator (SMS), serotonin antagonist and reuptake inhibitor (SARI), norepinephrine reuptake inhibitor (NRI), norepinephrine-dopamine reuptake inhibitor (NDRI), tricyclic antidepressant (TCA), tetracyclic antidepressant (TeCA), monoamine oxidase inhibitor (MAOI) and atypical antipsychotic.

31. The use of a salt, polymorph or composition according to any of claims 1-18 in the manufacture of medicament for the treatment of a disease or disorder according to any of claims 19-30.

32. A pharmaceutical formulation comprising a salt according to any of claim 1, 3, 4, 5, 7, 9, 10, 11, 13, 15, 16 or 17 and water.

33. The pharmaceutical composition according to any of claim 2, 8, 12 or 15, wherein said carrier is water.
